(19) United States
(12) Patent Application Publication
(10) Pub. No.: US 2005/0238721 A1
(43) Pub. Date: Oct. 27, 2005

(54) One step compounding extrusion of drug filled polymers

(76) Inventors: Lawrence A. Acquarulo JR., Lisbon, CT (US); Richard J. Quigley, Raynham, MA (US); Charles J. O'Neil, Chelmsford, MA (US); Anthony Listro, Woodstock, CT (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/101,383

(22) Filed: Apr. 7, 2005

Related U.S. Application Data
(60) Provisional application No. 60/560,341, filed on Apr. 7, 2004.

(51) Int. Cl. A61K 9/20; A61K 9/14

(52) U.S. Cl. 424/486; 264/165

(57) ABSTRACT

The present invention relates to a method for providing an article which contains a pharmaceutical ingredient and a polymeric material. The pharmaceutical ingredient may be melt compounded into the polymeric material and extruded through a profile die to provide a product.

### CROSS REFERENCE TO RELATED APPLICATIONS

The present application claims the benefit of the filing date of U.S. Provisional Application Ser. No. 60/560,341 filed Apr. 7, 2004, the teachings of which are incorporated herein by reference.

### FIELD OF INVENTION

The present invention relates to a method for providing an article including a polymeric material and a pharmaceutical ingredient. More particularly the present invention relates to a one-step process for melt compounding a pharmaceutical ingredient into a polymer material.

### BACKGROUND

Compounding of pharmaceutical ingredients into polymer materials has been performed for many years. A typical process involves using a compounding extruder, such as a high intensity twin screw mixer, to prepare a drug filled polymer. This process takes raw resins, drugs, and other excipients, fillers, additives, and colorants and meters them into the mixing extruder in the desired proportions. The ingredients are melt-mixed and extruded from the mixing extruder through a die suitable for making pellets either by strand pelletizing, hot die face, or under water pelletizing. Melt filtration may also be performed in this process. After the compounded polymer, drugs, etc. have been pelletized the pellets may be introduced to a single screw extruder and profiled extruded into a final desired product.

### SUMMARY

An embodiment of the present invention provides a method of preparing an article including a polymeric material and a pharmaceutical ingredient. The process herein includes the steps of introducing a polymeric material to a twin screw extruder and heating the polymeric material therein. A pharmaceutical ingredient may be metered into the twin screw extruder including the polymeric material in a desired concentration. The polymeric material and pharmaceutical ingredient are melt-compounded within the twin screw extruder. The melt-compounded polymeric material and pharmaceutical ingredient may then be extruded from the twin screw extruder through a profile extrusion die to achieve a final desired profile.

Another embodiment of the present invention relates to a method of preparing an article comprising supplying a polymeric material into an extruder; supplying a pharmaceutical ingredient and metering the pharmaceutical ingredient into the extruder. The pharmaceutical ingredient and polymer material are then melt compounded in the extruder to form a compound; and extruded through a profile extrusion die to obtain a final product.

Another embodiment of the present invention relates to a method for forming medical tubing or catheters including a medicament comprising supplying a polymeric material to a twin screw extruder; and providing a medicament. The medicament is introduced into the twin screw extruder; and melt-compounded with the polymeric material to form a compound. The compound is extruded through a profile die; and formed into medical tubing or catheter.

Another embodiment of the present invention relates to a method for forming films including a pharmaceutical ingredient comprising supplying a polymeric material to a twin screw extruder; providing a medicament; and introducing the medicament into the twin screw extruder. The medicament and the polymeric material are melt-compounded in the twin screw extruder to form a compound, extruded through a profile die, and formed into a film.

### DETAILED DESCRIPTION

The present invention is generally directed at a method for providing a polymeric material including a pharmaceutical ingredient. Consistent with the present invention a polymeric material may be melt extrusion-compounded with a drug or pharmaceutical ingredient using in a single extrusion process to form a final profile extruded product. The invention herein includes introducing a polymeric material to a twin screw compounding extruder. Selected pharmaceutical or bioactive ingredients may be metered into the twin screw extruder in a desired amount. The pharmaceutical ingredient and polymeric material may be melt-compounded in the twin screw extruder. The twin screw extruder may be equipped with an extrusion die having a geometry corresponding to a profile of a desired final product. After compounding, the mixture of polymeric material and pharmaceutical ingredient may be extruded through the profile extrusion die to produce a final product having a desired profile. Exemplary profiles may include tubes, films, fibers, etc.

While the description herein speaks to compounding a pharmaceutical ingredient and a polymeric material, it should be understood that various other ingredients, such as processing aids, fillers, modifiers, additives, etc. may also be incorporated into the mixture of polymer and pharmaceutical. Additionally, it should be understood that consistent with the present invention, pharmaceutical ingredient means one or more drugs, pharmaceutical products, and or bioactive ingredients.

Consistent with the present invention, the extrusion compounded polymer and pharmaceutical ingredients may be directly profile extruded from the twin screw compounding extruder into a desired final product. For example, the compounded polymer and pharmaceutical ingredient may be extruded to produce catheters, medical tubing, and medical film. Additionally, extruded products, such as films and fibers, may be subsequently processed to provide wound dressings, transdermal patches, and drug delivery devices. Such products may find application in areas such as veterinary medicine, consumer medical products, pharmaceutical cosmetics, medical devices, and military use.

By utilizing a one-step process, the number of handling steps of the pharmaceutical ingredient may be reduced. The reduction in the number of handling steps may not only reduce the risk of contamination of the pharmaceutical, but may also reduce the risk that harmful pharmaceuticals may pose to those handing the products.

Additionally, the one step process herein may reduce the heat history of the pharmaceutical ingredient. This may be particularly important for pharmaceutical ingredients having a special susceptibility to thermal degradation.

During conventional compounding, the melt mixed output of the compounding extruder may be drawn through a water bath to facilitate cooling of the extrudate. In order to prevent degradation of the polymer and/or the pharmaceutical ingredient the compounded mixture is probably dried prior to the step of single screw profile extrusion. Accordingly, utilizing a single-step compounding and extrusion process consistent with the present invention may also eliminate the need for intermediate drying of a pelletized material.

Mixing a pharmaceutical material with a polymeric material may have a significant impact on the processability. For example, a drug filled polymer may exhibit viscosity characteristics and lubricity that differ greater from the unfilled polymeric material. Therefore, from a processing stand point, the single step twin screw compounding and extrusion process herein avoids problems associated with processing a polymeric material filled with a pharmaceutical. Similarly, because a twin screw extruder utilized in carrying out the process of the present invention includes a positive conveying mechanism, variation in the feeding characteristics and melt rheology of the polymer melt and pharmaceutical ingredient may be easily accommodated without sacrificing uniform concentration of the pharmaceutical ingredient in the mixture.

According to one aspect the pharmaceutical ingredient may be provided in carefully controlled concentrations in the final product. By directly extruding a final product from a twin screw extruder, the concentration of the pharmaceutical in the final product may be within +/−10% of the intended level. That is, if the intended concentration of the pharmaceutical ingredient in the final product is 10% by weight of the total composition, the process according to the present invention may provide a concentration such that the actual concentration of the pharmaceutical ingredient may be provided within a window of 9-11% by weight of the total composition.

According to one embodiment, a twin screw extruder may be provided including a profile extrusion die. Consistent with one embodiment, the profile extrusion die may include a tube die. A thermoplastic polymeric material, such as ethylene vinyl acetate, may be introduced to the heated twin screw extruder and heated therein. A pharmaceutical ingredient may be metered into the twin screw extruding in an amount in the range of between 0.5% to 25% by weight of the final extruded product. More preferably the pharmaceutical ingredient may be metered into the twin screw extruder in an amount in the range of about 6 to 15% by weight of the final extruded product. The thermoplastic material may be melt-compounded with the pharmaceutical ingredient in the twin screw extruder. The melt-compounded thermoplastic material and pharmaceutical ingredient may then be extruded through the profile extrusion die to provide a tubular extrudate comprising the thermoplastic material including the pharmaceutical ingredient in the range between about 0.5% to 25% by weight of the tubular extrudate, and more preferably in the range of between about 6% to 15% by weight of the tubular extrudate.

The foregoing description is provided to illustrate and explain the present invention. However, the description hereinabove should not be considered to limit the scope of the invention set forth in the claims appended here to.

1. A method of preparing an article comprising:
supplying a polymeric material into an extruder;
supplying a pharmaceutical ingredient;
metering said pharmaceutical ingredient into said extruder;
melt compounding said pharmaceutical ingredient and said polymeric material in said extruder to form a compound; and
extruding said compound through a profile extrusion die to obtain a final product.

2. The method of claim 1, wherein said pharmaceutical ingredient includes a bioactive ingredient.

3. The method of claim 1, wherein said profile extrusion die shapes said compound into a geometry selected from the group consisting of tubes, films, fibers or combinations thereof.

4. The method of claim 3, wherein in said compound is shaped into a product selected from the group consisting of catheters, medical tubing, medical film and fibers.

5. The method of claim 1, further comprising supplying additional ingredients into said extruder selected from the group consisting of processing aids, fillers, modifiers, additives and combinations thereof.

6. The method of claim 1, wherein said pharmaceutical ingredient is included between 0.5-25% by weight of the compound.

7. The method of claim 1, wherein said polymer material includes ethylene vinyl acetate.

8. The method of claim 1, wherein said extruder is a twin screw extruder.

9. A method for forming medical tubing or catheters including a medicament comprising:
supplying a polymeric material to a twin screw extruder;
providing a medicament;
introducing said medicament into said twin screw extruder;
melt-compounding said medicament and said polymeric material in said twin screw extruder to form a compound;
extruding said compound through a profile die; and
forming said compound into medical tubing or catheter.

10. The method of claim 9, wherein said pharmaceutical ingredient includes a bioactive ingredient.

11. The method of claim 9, further comprising supplying additional ingredients into said twin screw extruder selected from the group consisting of processing aids, fillers, modifiers, additives and combinations thereof.

12. The method of claim 9, wherein said pharmaceutical ingredient is included between 0.5-25% by weight of the compound.

13. The method of claim 9, wherein said polymer material includes ethylene vinyl acetate.

14. A method for forming films including a pharmaceutical ingredient comprising:
supplying a polymeric material to a twin screw extruder;
providing a medicament;
introducing said medicament into said twin screw extruder;
melt-compounding said medicament and said polymeric material in said twin screw extruder to form a compound;
extruding said compound through a profile die; and
forming said compound into a film.

15. The method of claim 14, wherein said pharmaceutical ingredient includes a bioactive ingredient.

16. The method of claim 14, further comprising supplying additional ingredients selected from the group consisting of processing aids, fillers, modifiers, additives and combinations thereof.

17. The method of claim 14, wherein said pharmaceutical ingredient is included between 0.5-25% by weight of the compound.

18. The method of claim 14, wherein said polymer material includes ethylene vinyl acetate.
